(19) United States
(12) Patent Application Publication
(10) Pub. No.: US 2025/0297976 A1
(43) Pub. Date: Sep. 25, 2025

(54) GAS SENSOR

(71) Applicant: TDK Corporation, Tokyo (JP)

(72) Inventor: Kei TANABE, Tokyo (JP)

(21) Appl. No.: 19/066,874

(22) Filed: Feb. 28, 2025

(30) Foreign Application Priority Data
Mar. 19, 2024 (JP) 2024-043386

(51) Int. Cl. G01N 25/18; G01N 33/00

(52) U.S. Cl. CPC G01N 25/18; G01N 33/0027

(57) ABSTRACT

Disclosed herein is a gas sensor that includes: a sensor part configured to generate a detection signal corresponding to a concentration of a gas to be detected; and a control circuit configured to calculate, based on the detection signal, an output signal indicating the concentration of the gas to be detected. The sensor part includes a temperature-sensitive element and a heater configured to heat the temperature-sensitive element. The control circuit is configured to correct the output signal or change a heating condition of the heater in accordance with a flow velocity signal indicating gas flow velocity in measuring atmosphere.

### CROSS-REFERENCE TO RELATED APPLICATION

This application claims the benefit of Japanese Patent Application No. 2024-043386, filed on Mar. 19, 2024, the entire disclosure of which is incorporated by reference herein.

### BACKGROUND OF THE ART

### Field of the Art

The present disclosure relates to a gas sensor and, more particularly, to a gas sensor capable of accurately measuring gas concentration irrespective of gas flow velocity in measuring atmosphere.

### Description of Related Art

Japanese Patent No. 7,070,175 discloses a gas sensor capable of reducing a measurement error caused by a gas different from a gas to be detected.

### SUMMARY

A gas sensor according to an aspect of the present disclosure includes a sensor part configured to generate a detection signal corresponding to a concentration of a gas to be detected and a control circuit configured to calculate, based on the detection signal, an output signal indicating the concentration of the gas to be detected. The sensor part includes a temperature-sensitive element and a heater configured to heat the temperature-sensitive element. The control circuit is configured to correct the output signal or change a heating condition of the heater in accordance with a flow velocity signal indicating gas flow velocity in measuring atmosphere.

### BRIEF DESCRIPTION OF THE DRAWINGS

The above features and advantages of the present disclosure will be more apparent from the following description of some embodiments taken in conjunction with the accompanying drawings, in which:

FIG. 1 is a circuit diagram illustrating the configuration of a gas sensor 100 according to a first embodiment of the technology described herein;

FIGS. 2A and 2B are circuit examples of the reference voltage generating circuit 32;

FIG. 3 is a graph for explaining the influence that gas flow velocity in measuring atmosphere has on measurement results;

FIG. 4 is a circuit diagram illustrating the configuration of a gas sensor 200 according to a second embodiment of the technology described herein;

FIG. 5 is a timing chart for explaining a method of correcting the heater voltages V13 and V14 using the heater voltage correction table 35b;

FIG. 6 is a circuit diagram illustrating the configuration of a gas sensor 300 according to a third embodiment of the technology described herein; and

FIG. 7 is a timing chart for explaining a method of correcting the heating times using the heating time correction table 35c.

### DETAILED DESCRIPTION OF THE EMBODIMENTS

The present inventor has found that a measurement error is caused due to gas flow velocity in measuring atmosphere.

The present disclosure describes a technology relating to a gas sensor capable of accurately measuring gas concentration irrespective of gas flow velocity in measuring atmosphere.

Some embodiments of the present disclosure will be explained below in detail with reference to the accompanying drawings.

### First Embodiment

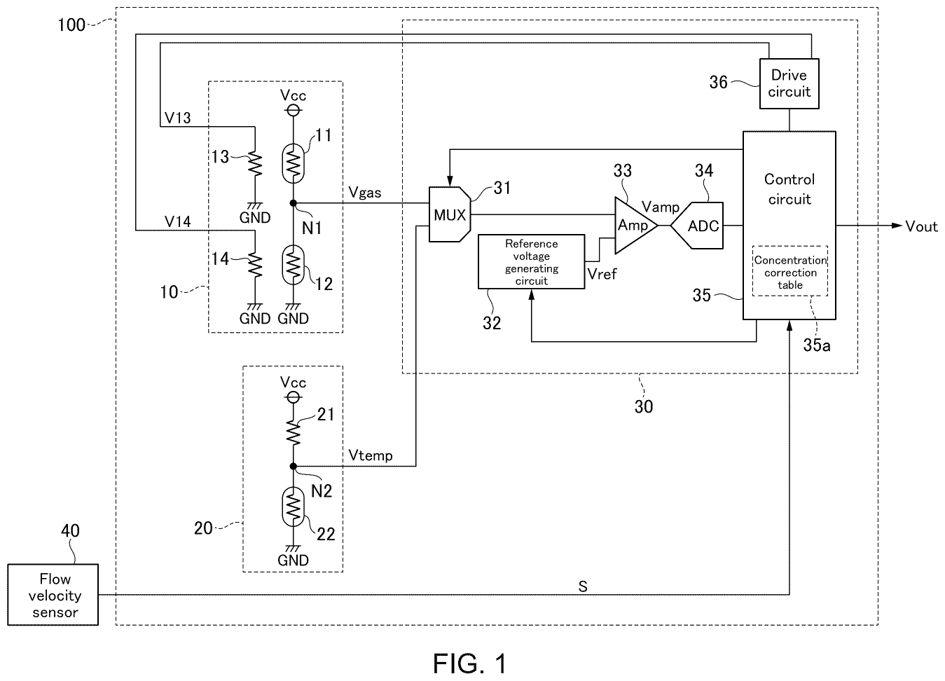

FIG. 1 is a circuit diagram illustrating the configuration of a gas sensor 100 according to a first embodiment of the technology described herein.

As illustrated in FIG. 1, the gas sensor 100 according to the first embodiment includes a sensor part 10 that generates a detection signal Vgas corresponding to the concentration of a gas to be detected, a temperature sensor 20 that generates a temperature signal Vtemp corresponding to environmental temperature, and a signal processing circuit 30. Although not particularly limited, the gas sensor 100 according to the present embodiment is a heat-conduction type gas sensor for detecting the concentration of CO₂ gas in measuring atmosphere.

The sensor part 10 includes thermistors 11 and 12 connected in series between a power supply Vcc and a ground GND and heaters 13 and 14 for heating the thermistors 11 and 12, respectively. The detection signal Vgas output from the sensor part 10 appears at a node N1 between the thermistors 11 and 12. The thermistor 11 is a temperature-sensitive element for detection, while the thermistor 12 is a temperature-sensitive element for reference. The thermistors 11 and 12 are resistors whose reference value changes with temperature. Examples of the material of the thermistors 11, 12 and a thermistor 22 to be described later include vanadium oxide, amorphous silicon, polycrystalline silicon, an oxide with a spinel crystal structure containing manganese, titanium oxide, and yttrium-barium-copper oxide.

When CO₂ gas is present in measuring atmosphere in a state where the thermistor 11 as the temperature-sensitive element for detection is heated in the range of 100° C. to 230° C. (e.g., to around 150° C.) that is a temperature zone exhibiting high CO₂ gas detection sensitivity, heat dissipation characteristics of the thermistor 11 change in accordance with the concentration of CO₂ gas. This change appears as a change in the temperature of the thermistor 11, i.e., a change in the resistance value thereof. CO₂ gas is lower in heat dissipation than air, so that the temperature of the thermistor 11 increases as the concentration of CO₂ gas becomes high. Thus, in a case where heating is performed so that the temperature of the thermistor 11 becomes 150° C. in measuring atmosphere where CO₂ gas concentration is, for example, zero, if CO₂ gas is present in measuring atmosphere, the temperature of the thermistor 11 increases with an increase in CO₂ gas concentration and exceeds 150° C. As a result, the resistance value of the thermistor 11 lowers as CO₂ gas concentration in measuring atmosphere increases.

On the other hand, even when CO₂ gas is present in measuring atmosphere in a state where the thermistor 12 as the temperature-sensitive element for reference is heated in the range of 300° C. to 450° C. (e.g., to around 300° C.) that is a temperature zone exhibiting low CO₂ gas detection sensitivity, the heat dissipation characteristics of the thermistor 12 hardly change in accordance with the concentration of CO₂ gas, with the result that the temperature of the thermistor 12 hardly changes. Accordingly, a CO₂ gas concentration-dependent change in the resistance value of the thermistor 12 heated to around 300° C. is sufficiently smaller than a CO₂ gas concentration-dependent change in the resistance value of the thermistor 11 heated to around 150° C. There may be almost no CO₂ gas concentration-dependent change in the resistance value of the thermistor 12 heated to around 300° C. As a result, when the thermistors 11 and 12 are heated to around 150° C. and around 300° C., respectively (when heating is performed so that temperatures of the thermistors 11 and 12 become 150° C. and 300° C., respectively, in measuring atmosphere where CO₂ gas concentration is, for example, zero), the detection signal Vgas corresponding to the concentration of CO₂ gas in measuring atmosphere appears at the node N1 between the thermistors 11 and 12. On the other hand, even when another gas, in which there is no significant difference between heat dissipation characteristics exhibited when the thermistor 11 is heated to around 150° C. and those exhibited when the thermistor 12 is heated to around 300° C., is contained in measuring atmosphere, the concentration of this gas has little influence on the detection signal Vgas. This allows the sensor part 10 to selectively detect the concentration of CO₂ gas.

The temperature sensor 20 includes a resistor 21 and a thermistor 22 which are connected in series between the power supply Vcc and the ground GND. A temperature signal Vtemp of the temperature sensor 20 appears at a node N2 between the resistor 21 and the thermistor 22. The temperature sensor 20 detects environmental temperature. Environmental temperature is a temperature in measuring atmosphere. The temperature sensor 20 may be designed so as not to be affected or so as to be hardly affected by heating by, for example, the heaters 13 and 14.

The signal processing circuit 30 includes a multiplexer 31, a reference voltage generation circuit 32, a differential amplifier 33, an AD converter (ADC) 34, a control circuit 35, and a drive circuit 36.

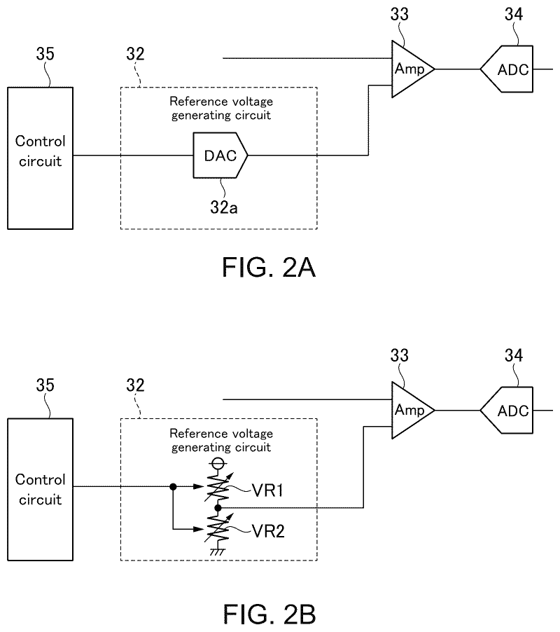

The multiplexer 31 supplies one of the detection signal Vgas and temperature signal Vtemp to the differential amplifier 33 under the control of the control circuit 35. The differential amplifier 33 generates an amplified signal Vamp which is a signal obtained by amplifying the difference (potential difference) between the level of one of the detection signal Vgas and temperature signal Vtemp and the level of a reference signal Vref generated by the reference voltage generation circuit 32. The reference voltage generation circuit 32 may be constituted by a DA converter 32a (FIG. 2A) that D-A converts a digital value output from the control circuit 35 or by variable resistors VR1 and VR2 (FIG. 2B) whose resistance values are controlled by the control circuit 35.

The amplified signal Vamp output from the differential amplifier 33 is input to the AD converter 34. The AD converter 34 A-D converts the amplified signal Vamp to generate a digital value and supplies the generated digital value to the control circuit 35.

The control circuit 35 calculates the concentration of CO₂ gas which is a gas to be detected based on the amplified signal Vamp of the detection signal Vgas and generates an output signal Vout indicating the CO₂ gas concentration. The control circuit 35 calculates the CO₂ gas concentration using a calculation formula set therein. Further, the control circuit 35 controls, through the drive circuit 36, the levels of the heater voltages V13 and V14 to be supplied to the heaters 13 and 14, respectively.

The control circuit 35 corrects the heater voltages V13 and V14 in accordance with the amplified signal Vamp of the temperature signal Vtemp. For example, the control circuit 35 makes the heaters 13 and 14 heat the thermistors 11 and 12 for a predetermined period of time to correct the heater voltages V13 and V14 so that the temperatures of the thermistors 11 and 12 become 150° C. and 300° C., respectively, irrespective of environmental temperature in cases where gas flow velocity in measuring atmosphere is zero and the concentration of CO₂ gas in measuring atmosphere is, for example, zero. That is, the control circuit 35 changes the levels of the heater voltages V13 and V14 in accordance with the temperature signal Vtemp (amplified signal Vamp) to change power to be applied to the heaters 13 and 14, thereby changing heat generation amounts of the heaters 13 and 14.

Further, the control circuit 35 corrects the output signal Vout in accordance with a flow velocity signal S supplied from a flow velocity sensor 40. The flow velocity sensor 40 may be a device constituting a part of the gas sensor 100 or a device provided outside the gas sensor 100. The control circuit 35 corrects the output signal Vout with reference to a concentration correction table 35a set up in the control circuit 35. The concentration correction table 35a is a data table indicating the relationship between the flow velocity signal S and a correction amount required for the output signal Vout. The flow velocity signal S is a signal indicating gas flow velocity in measuring atmosphere. The higher the flow velocity the flow velocity signal S indicates, the lower the heating temperatures of the thermistors 11 and 12 become, and the control circuit 35 corrects this.

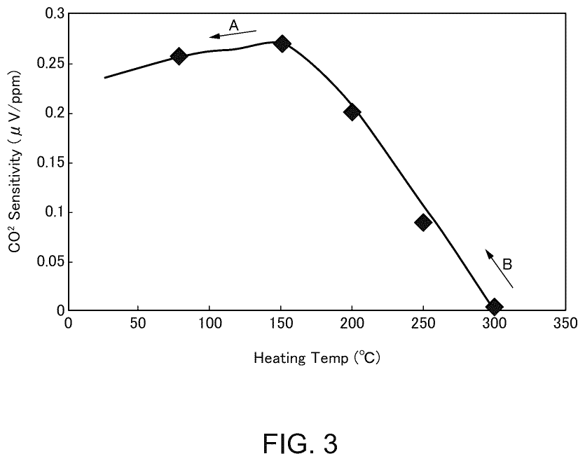

FIG. 3 is a graph for explaining the influence that gas flow velocity in measuring atmosphere has on measurement results and illustrates the relationship between heating temperatures and detection sensitivities of the thermistor 11 and 12.

As illustrated in FIG. 3, the relationship between the detection sensitivities of the thermistors 11 and 12, i.e., CO₂ gas concentration in measuring atmosphere and resistance values of the thermistors 11 and 12 significantly changes depending on the heating temperatures of the thermistors 11 and 12. That is, the CO₂ gas detection sensitivities of the thermistors 11 and 12 become maximum at about 150° C., whereas they become substantially zero in a temperature range equal to or higher than 300° C. Thus, as described above, it is possible to selectively detect the concentration of CO₂ gas by heating the thermistors 11 and 12 to about 150° C. and about 300° C., respectively.

However, when gas flow occurs in measuring atmosphere, the thermistors 11 and 12 are cooled by the gas flow, and thus the heating temperatures thereof decrease. Thus, as the flow velocity increases, the heating temperature of the thermistor 11 decreases as denoted by the arrow A in FIG. 3, so that the heating temperature becomes less than 150° C. to lower the detection sensitivity. As a result, the change amount of the detection signal Vgas with respect to a change in CO₂ gas concentration decreases. Further, as the flow velocity increases, the heating temperature of the thermistor 12 decreases as denoted by the arrow B in FIG. 3, so that the heating temperature becomes less than 300° C. to increase the detection sensitivity. As a result, a change occurs in the resistance value of the thermistor 12 depending on the concentration of CO₂ gas. This also reduces the change amount of the detection signal Vgas with respect to a change in CO₂ gas concentration. As described above, when gas flow occurs in measuring atmosphere, the detection signal Vgas changes due to the velocity of the gas flow.

The control circuit 35 uses the concentration correction table 35a to correct the output signal Vout so as to cancel a measurement error caused due to such flow velocity. The correction amount of the output signal Vout is determined based on the flow velocity signal S. The higher the flow velocity the flow velocity signal S indicates, the larger the correction amount of the output signal Vout. This enables accurate detection of CO₂ gas concentration irrespective of the gas flow velocity in measuring atmosphere.

Further, the control circuit 35 may change the level of the reference signal Vref in accordance with the flow velocity signal S. Thus, even if an offset occurs in the midpoint level (the level of the detection signal Vgas appearing at the node N1 when CO₂ gas concentration in measuring atmosphere is, for zero) the example, of detection signal Vgas due to gas flow velocity in measuring atmosphere, it can be canceled, thus preventing reduction in dynamic range.

As described above, the gas sensor 100 according to the first embodiment corrects the output signal Vout in accordance with the flow velocity signal S and can thus accurately detect CO₂ gas concentration irrespective of gas flow velocity in measuring atmosphere.

The measurement error caused due to gas flow velocity in measuring atmosphere can be canceled by changing heating conditions of the heaters 13 and 14. The following describes an embodiment that cancels the measurement error caused due to gas flow velocity in measuring atmosphere by changing heating conditions of the heaters 13 and 14.

### Second Embodiment

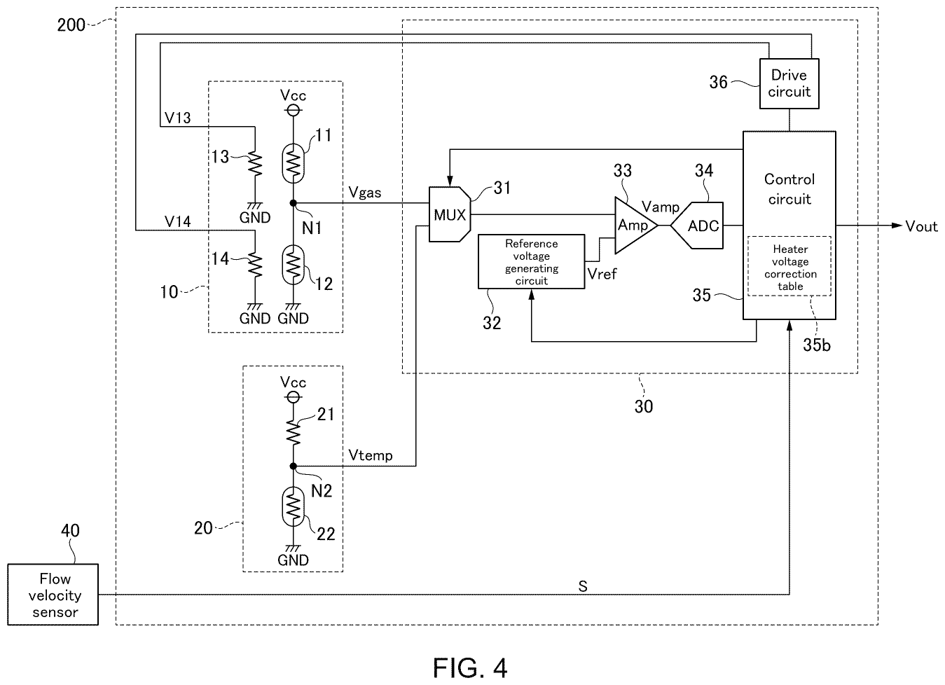

FIG. 4 is a circuit diagram illustrating the configuration of a gas sensor 200 according to a second embodiment of the technology described herein.

As illustrated in FIG. 4, the gas sensor 200 according to the second embodiment differs from the gas sensor 100 according to the first embodiment in that the control circuit 35 includes a heater voltage correction table 35b in place of the concentration correction table 35a. Other basic configurations are the same as those of the gas sensor 100 according to the first embodiment, so the same reference numerals are given to the same elements, and overlapping description will be omitted.

The control circuit 35 uses the heater voltage correction table 35b to correct the levels of the heater voltages V13 and V14 so as to cancel a measurement error caused due to gas flow velocity in measuring atmosphere. The heater voltage correction table 35b is a data table indicating the relationship between the flow velocity signal S and the correction amounts of the heater voltages V13 and V14.

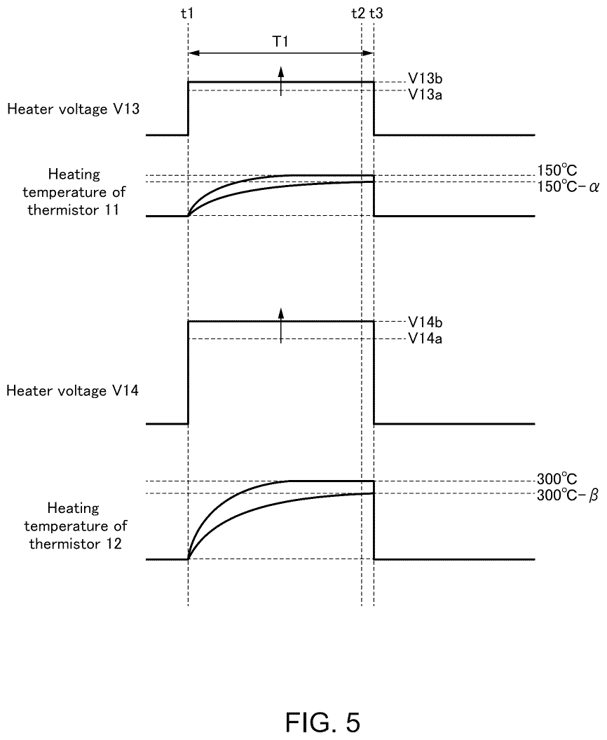

FIG. 5 is a timing chart for explaining a method of correcting the heater voltages V13 and V14 using the heater voltage correction table 35b.

In the example illustrated in FIG. 5, the heater voltages V13 and V14 are applied to the heaters 13 and 14, respectively, in the period T1 from time t1 to time t3. The level V13a illustrated in FIG. 5 is the level of the heater voltage V13 when gas flow velocity in measuring atmosphere is zero, and the level V14a illustrated in FIG. 5 is the level of the heater voltage V14 when gas flow velocity in measuring atmosphere is zero. That is, when the levels of the heater voltages V13 and V14 are set to V13a and V14a, respectively, under the condition that gas flow velocity in measuring atmosphere is zero, the heating temperatures of the thermistors 11 and 12 become about 150° C. and about 300° C., respectively (the heating temperatures of the thermistors 11 and 12 become 150° C. and 300° C., respectively, under the condition that CO₂ gas concentration in measuring atmosphere is, for example, zero).

However, when the heater voltages V13 and V14 are set to V13a and V14a, respectively, in the presence of gas flow in measuring atmosphere, the thermistors 11 and 12 are cooled by gas flow, and the heating temperatures thereof decrease to 150° C.-α and 300° C.-β, respectively. The control circuit 35 sets the levels of the heater voltages V13 and V14 to V13b (>V13a) and V14b (>14a), respectively, in accordance with the flow velocity signal S so as to cancel such decreases in the heating temperatures. The level V13b of the heater voltage V13 is obtained by reading a correction amount corresponding to the flow velocity signal S from the heater voltage correction table 35b and adding the correction amount to the level V13a, and the level V14b of the heater voltage V14 is obtained by reading a correction amount corresponding to the flow velocity signal S from the heater voltage correction table 35b and adding the correction amount to the level V14a. Thus, even when gas flow occurs in measuring atmosphere, the thermistors 11 and 12 are properly heated to about 150° C. and about 300° C., respectively. Then, by sampling the detection signal Vgas at time t2 immediately before time t3, it is possible to accurately measure gas concentration.

As described above, the gas sensor 200 according to the second embodiment changes the levels of the heater voltages V13 and V14 in accordance with the flow velocity signal S to change power to be applied to the heaters 13 and 14, thereby changing heat generation amounts of the heaters 13 and 14. The correction amounts of the heater voltages V13 and V14 are determined based on the flow velocity signal S. The higher the flow velocity the flow velocity signal S indicates, the larger the correction amount of the levels of the heater voltages V13 and V14. This enables accurate detection of CO₂ gas concentration irrespective of the gas flow velocity in measuring atmosphere.

### Third Embodiment

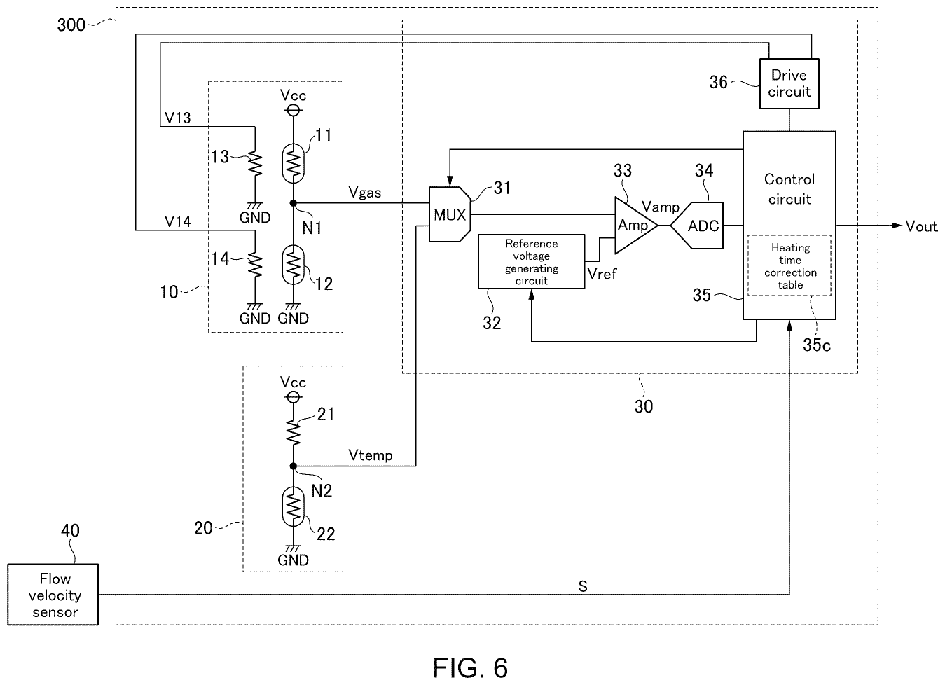

FIG. 6 is a circuit diagram illustrating the configuration of a gas sensor 300 according to a third embodiment of the technology described herein.

As illustrated in FIG. 6, the gas sensor 300 according to the third embodiment differs from the gas sensor 100 according to the first embodiment in that the control circuit 35 includes a heating time correction table 35c in place of the concentration correction table 35a. Other basic configurations are the same as those of the gas sensor 100 according to the first embodiment, so the same reference numerals are given to the same elements, and overlapping description will be omitted.

The control circuit 35 uses the heating time correction table 35c to correct application times of the heater voltages V13 and V14 so as to cancel a measurement error caused due to gas flow velocity in measuring atmosphere. The heating time correction table 35c is a data table indicating the relationship between the flow velocity signal S and the application times of the heater voltages V13 and V14.

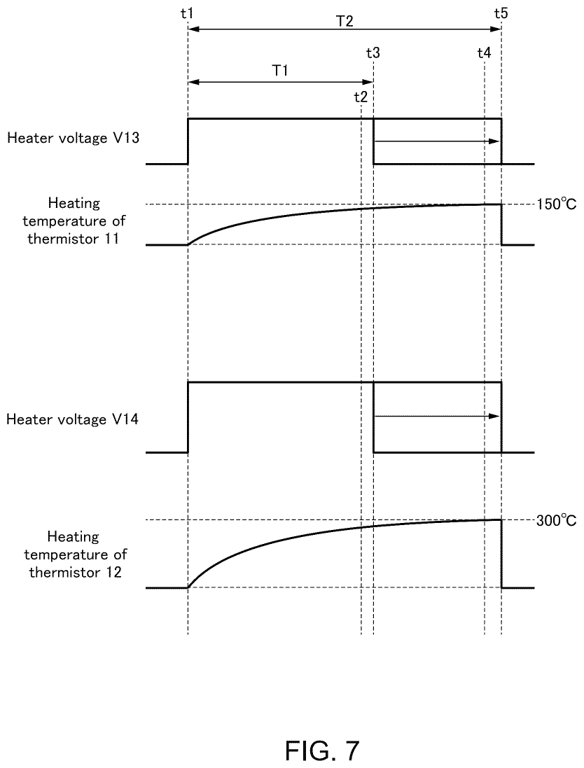

FIG. 7 is a timing chart for explaining a method of correcting the heating times using the heating time correction table 35c.

In the example illustrated in FIG. 7, the heater voltages V13 and V14 are applied to the heaters 13 and 14, respectively, in the period T1 from time t1 to time t3 when gas flow velocity in measuring atmosphere is zero. When gas flow velocity in measuring atmosphere is zero, the heating temperatures of the thermistors 11 and 12 reach about 150° C. and about 300° C., respectively, at time t2 immediately before time t3 (the heating temperatures of the thermistors 11 and 12 become 150° C. and 300° C., respectively, under the condition that CO₂ gas concentration in measuring atmosphere is, for example, zero). Thus, by sampling the detection signal Vgas at time t2, it is possible to obtain an accurate measurement of gas concentration.

However, when gas flow occurs in measuring atmosphere, the thermistors 11 and 12 are cooled by the gas flow, and thus the heating temperatures thereof do not reach 150° C. and 300° C., respectively, even at time t2. The control circuit 35 increases, according to the flow velocity signal S, the application times of the heater voltages V13 and V14 to the period T2 (>T1) from time t1 to time t5 so as to make up for such a shortage of the heating times. Thus, even when gas flow occurs in measuring atmosphere, the thermistors 11 and 12 are properly heated to about 150° C. and about 300° C., respectively. Then, by sampling the detection signal Vgas at time t4 immediately before time t5, it is possible to obtain an accurate measurement of gas concentration.

As described above, the gas sensor 300 according to the third embodiment changes the application times of the heater voltages V13 and V14 in accordance with the flow velocity signal S to change the heating times of the heaters 13 and 14. The application times of the heater voltages V13 and V14 are determined based on the flow velocity signal S. The higher the flow velocity the flow velocity signal S indicates, the longer the application times of the heater voltages V13 and V14. This enables accurate detection of CO₂ gas concentration irrespective of the gas flow velocity in measuring atmosphere.

While some embodiments of the technology according to the present disclosure have been described, the technology according to the present disclosure is not limited to the above embodiments, and various modifications may be made within the scope of the present disclosure, and all such modifications are included in the technology according to the present disclosure.

For example, although a thermistor serving as a resistor is used as a temperature-sensitive element of the sensor part 10 in the above embodiments, the present invention is not limited to this. For example, platinum (Pt) or tungsten (W) serving as a resistor may be used as the temperature-sensitive element.

Further, the levels of the heater voltages V13 and V14 are changed in accordance with the flow velocity signal S in the second embodiment, and the application times of the heater voltages V13 and V14 are changed in accordance with the flow velocity signal S; however, both the levels and application times of the heater voltages V13 and V14 may be changed in accordance with the flow velocity signal S.

The technology according to the present disclosure includes the following configuration examples, but not limited thereto.

A gas sensor according to an aspect of the present disclosure includes a sensor part that generates a detection signal corresponding to the concentration of a gas to be detected and a control circuit that calculates, based on the detection signal, an output signal indicating the concentration of the gas to be detected. The sensor part includes a temperature-sensitive element and a heater for heating the temperature-sensitive element. The control circuit corrects the output signal or changes a heating condition of the heater in accordance with a flow velocity signal indicating gas flow velocity in measuring atmosphere. This makes it possible to accurately detect gas concentration irrespective of gas flow velocity in measuring atmosphere.

In the above gas sensor, the control circuit may have a concentration correction table and correct the output signal with reference to the concentration correction table in accordance with the flow velocity signal. This makes it possible to properly correct the output signal in accordance with gas flow velocity in measuring atmosphere.

The above gas sensor may further include a differential amplifier that amplifies the potential difference between the detection signal and a reference signal, and the control circuit may change the level of the reference signal in accordance with the flow velocity signal. This makes it possible to ensure a sufficient dynamic range.

In the above gas sensor, the control circuit may change power to be applied to the heater in accordance with the flow velocity signal. This makes it possible to heat the temperature-sensitive element to a desired temperature even when gas flow occurs in measuring atmosphere.

In the above gas sensor, the control circuit may change heating time of the heater in accordance with the flow velocity signal. This makes it possible to heat the temperature-sensitive element to a desired temperature even when gas flow occurs in measuring atmosphere.

The above gas sensor may further include a temperature sensor that generates a temperature signal in accordance with environmental temperature, and the control circuit may change power to be applied to the heater in accordance with the temperature signal. This makes it possible to heat the temperature-sensitive element to a desired temperature irrespective of environmental temperature.

What is claimed is:

1. A gas sensor comprising:
a sensor part configured to generate a detection signal corresponding to a concentration of a gas to be detected; and
a control circuit configured to calculate, based on the detection signal, an output signal indicating the concentration of the gas to be detected,
wherein the sensor part includes a temperature-sensitive element and a heater configured to heat the temperature-sensitive element, and
wherein the control circuit is configured to correct the output signal or change a heating condition of the heater in accordance with a flow velocity signal indicating gas flow velocity in measuring atmosphere.

2. The gas sensor as claimed in claim 1,
wherein the control circuit has a concentration correction table, and
wherein the control circuit is configured to correct the output signal with reference to the concentration correction table in accordance with the flow velocity signal.

3. The gas sensor as claimed in claim 1, further comprising a differential amplifier configured to amplify a potential difference between the detection signal and a reference signal,
wherein the control circuit is configured to change a level of the reference signal in accordance with the flow velocity signal.

4. The gas sensor as claimed in claim 1, wherein the control circuit is configured to change power to be applied to the heater in accordance with the flow velocity signal.

5. The gas sensor as claimed in claim 1, wherein the control circuit is configured to change heating time of the heater in accordance with the flow velocity signal.

6. The gas sensor as claimed in claim 1, further comprising a temperature sensor configured to generate a temperature signal in accordance with environmental temperature,
wherein the control circuit is configured to change power to be applied to the heater in accordance with the temperature signal.
